(19) United States
(12) Patent Application Publication
(10) Pub. No.: US 2005/0261694 A1
(43) Pub. Date: Nov. 24, 2005

(54) Assembly for use in orthopaedic surgery

(76) Inventors: Marcus Orton, Boston Spa (GB); Gary Moore, Wetherby (GB)

Correspondence Address: Paul J Maginot;Maginot Moore & Beck, Bank One Center/Tower, Suite 3000 111 Monument Circle, Indianapolis, IN, 46204-5115, US

(21) Appl. No.: 10/513,562

(22) PCT Filed: May 6, 2003
(86) PCT No.: PCT/GB03/01914

§ 371 Date: May 16, 2005

(30) Foreign Application Priority Data
May 7, 2002 (GB) 0210362.0

(51) Int. Cl. A61B 17/32

(52) U.S. Cl. 606/81

(57) ABSTRACT

An assembly for use in orthopaedic surgery, comprises a component (2, 34, 50) which is to be positioned within a body cavity to engage a bone. The component comprises a hollow shell which is open on one side to allow access to its interior and has a fastening formation (10, 12, 14, 16, 18, 20, 60) within it. The assembly includes a manipulator (36, 52) having a clasp (62) for engaging the fastening formation so as to fasten the component to the manipulator. The portion of the formation which is engaged by the clasp is located within the shell so that, when the component is fastened to the manipulator, the clasp is located at least partially within the shell.

This invention relates to an assembly for use in orthopaedic surgery, which comprises a hollow shell component having a bar extending across it, and a manipulator having a clasp for engaging the bar so as to fasten the component to the manipulator.

Hollow shell components have uses in orthopaedic surgery such as shaping bone tissue, for example to receive a component of an orthopaedic joint prosthesis, and as orthopaedic joint prosthesis components. An instrument having a hollow shell configuration has to be manipulated when used to shape a bone. For example, when the instrument is a cutting tool with cutting teeth on its external surface, it can be rotated about an axis of symmetry (for example when the external surface defines part of a sphere) to cause the bone tissue to be cut. A component of a joint prosthesis has to be manipulated to ensure that it is aligned properly with the prepared surface of the bone.

It is known to provide a hollow shell component with a bar which extends across it which can be engaged by a manipulator with an appropriate clasp. In the case of an instrument, the bar does not need to be detached from the shell, and can be bonded to the shell or formed integrally with it, for example by casting. In the case of a component of a joint prosthesis, the bar can be attached to the shell component by means of appropriate formations, for example which engage a lip on the component.

It is desirable to minimise the size of the incision that is necessary during surgery, for example to minimise blood loss and damage to soft tissue, as well as for aesthetic reasons. When performing surgery on a patient's hip joint, especially when implanting an acetabular cup prosthesis, it is generally the case that the incision has to be capable of accommodating the cup prosthesis itself when directed towards the acetabulum, aligned appropriately relative to the relevant axis.

The present invention provides an assembly for use in orthopaedic surgery which comprises a shell component and a manipulator, in which the shell has a bar extending across it located within the shell, so that a clasp on the manipulator which engages the bar is located within the shell.

Accordingly, in one aspect, the invention provides an assembly for use in orthopaedic surgery, which comprises a component which is to be positioned within a body cavity to engage a bone, the component comprising a hollow shell which is open on one side to allow access to its interior and which has a fastening formation within it, and a manipulator having a clasp for engaging the fastening formation so as to fasten the component to the manipulator, in which the portion of the formation which is engaged by the clasp is located within the shell so that, when the component is fastened to the manipulator, the clasp is located at least partially within the shell.

The assembly of the present invention has the advantage that, for a given size of shell component and a given manipulator, the overall size of the assembly is smaller than the size of known assemblies in which the fastening formation is located at the open side of the shell component. In particular, it can allow a surgical procedure to be performed through a smaller incision than might be necessary using known assemblies.

The external surface of the shell can be symmetrical about an axis of rotation. For example, the external surface can define a part of a sphere. This might be the case when the assembly is for use in connection with a joint prosthesis in which one component articulates against the other component in the manner of a ball which is received in a cup. While the configuration of the external surface can preferably be symmetrical about an axis of rotation, the component need not be symmetrical in this way. For example, the shell can have one or more cut-out portions. The wall thickness of the shell can vary from one region to another. The shell can have features on its outer surface according to its intended purpose: for example, the component might be a cutting tool, in which case it can have cutting teeth on its outer surface, and the arrangement of the teeth on the surface need not necessarily be symmetrical about the axis of symmetry.

When the component is a cutting tool, the use of a rotationally symmetrical component has the advantage that the tool can be rotated about the axis to cut the patient's bone tissue.

When the external surface of the component defines a part of a sphere, the tool can be rotated about its axis to cut the patient's bone tissue, while at the same time the orientation of the axis relative to the bone is changed. This can be important to achieve satisfactory cutting of the patient's bone in preparation for implantation of a joint prosthesis component.

Preferably, the fastening formation to which the clasp fastens intersects the axis of rotational symmetry at the point where it is engaged by the clasp.

Preferably, the ratio of the depth of the fastening formation within the shell measured from the open side of the shell, to the length of the axis measured from the open side of the shell to the external surface of the shell opposite the open side, is at least about 0.2. When the fastening formation intersects the axis of symmetry of the component, the depth of the formation is measured from the open side of the shell to the point where the centre of the formation intersects the axis of symmetry. When the formation does not intersect the axis of symmetry, the depth of the formation is measured along the axis, to the point at which it is engaged by the clasp. Preferably, the value of the said ratio is at least about 0.4, more preferably at least about 0.5.

Preferably, the fastening formation is a bar which extends across the shell. The bar can be straight (when viewed along a line perpendicular to the axis of the shell and to the bar), in which case, it will be fastened to the internal wall of the shell at its ends, at the same depth as point at which the clasp engages the bar. The bar can be cranked, so that the depth of the ends of the bar within the shell need not be the same as the depth of the point at which the clasp engages the bar. For example, the ends of the bar can be located closer to the open side of the shell than the point at which the clasp engages the bar, for example with the ends of the bar fastened to the shell at the open side. Preferably, the bar is approximately straight in the region thereof in which it is engaged by the clasp. The ends of the bar can be located further from the open side of the shell than the point at which the clasp engages the bar; for example the ends of the bar can be fastened to the internal surface of the shell close to the pole of the shell. The bar can also be mounted on a fixture which is fastened to the internal wall of the shell at or close to the pole: for example, the fixture can comprise a length of a tube, and the bar extends across the tube.

The bar is preferably straight when viewed along the axis of the shell (which is axis of symmetry when the shell is rotationally symmetrical). A bar can be considered as consisting of a plurality of limbs extending from the polar axis of the component: for example, a single bar which extends across the component from one side to the opposite side consists of two limbs. The bar can include more than two limbs or there can be more than one bar. For example, there can be two bars which are fastened together at about the shell axis, so that there are four limbs extending radially from the axis. The bars can then be perpendicular to one another at the point at which they are fastened together. Other arrangements are envisaged, for example in which the bar is provided by three limbs which are joined together so that the angle between any two of the limbs is about 120°. The limbs can be joined together at or close to the axis of the component. They might however not extend to the axis of the component and be joined together by means of a ring which encircles the axis.

Preferably, the clasp comprises a recess which is shaped to receive the bar, and a locking part which can be moved between two positions in which (a) the bar is prevented from moving out of the recess, and (b) the bar can be moved out of the recess, respectively. Preferably, the clamp defines at least one recess which can present a transverse opening which allows the bar to be positioned in the recess by sliding it transversely into the recess. The clamp can provide a plurality of recesses, corresponding to each limb of the bar or bars: for example, when there is one bar which extends across the component,

For example, the recess can be approximately C-shaped, so that the bar is received in the recess by being slid transversely relative to the axis of the component. The transverse sliding of the bar can involve relative translocation of the whole component relative to the clasp, or relative rotation (especially without translocation) between the component and the clasp in a plane which is transverse to the axis of the component, or both.

A retractable pin can close a recess, in which retraction of the pin allows the bar to be moved out of the recess. The pin can be profiled so that it can be displaced by the bar when force is applied to the bar to force it into the recess.

Preferably, the clasp includes at least two recesses. For example, when the bar extends across the shell, the clasp can include two recesses to engage the bar on opposite sides of the centre. When the bar has three limbs, the clasp can present three recesses. The recesses can be arranged so that the clasp is rotationally symmetrical, facilitating engagement of the component with the clasp by relative rotation. Preferably, each recess has a respective locking part.

Preferably the movable locking parts of the clasp which cooperate with respective recesses to engage the bar can be moved together between the locked and released positions. For example each of the locking parts can be provided on a sliding collar.

Preferably, the bar can allow rotation of the bar relative to the shaft. This can conveniently be achieved using a bar which has a generally rounded cross-section. Preferably, the clasp has a generally rounded recess in which the bar can be received.

Preferably, the clasp has a lock which can engage the bar to restrict rotation of the bar relative to the clasp. Preferably, the lock can be moved between a first position in which the bar can rotate within the clasp and a second position in which rotation of the bar is restricted. For example, the bar can have at least one aperture in it, and the clasp can include a retractable pin which can be received in the aperture in the bar to lock the bar against rotation relative to the clasp. The bar can have a flat on one side (or more than one flat, for example two flats on opposite sides) and the lock can comprise a C-shaped collar which a flat side, which can only fit on to the bar when the flat on the bar and the flat side on the collar are aligned. Preferably, the lock is biassed towards the position in which it restricts rotation of the bar. The lock (for example the pin or the C-shaped collar) can be mounted on a collar which can slide relative to the clasp. The lock can be provided on the same collar as locking parts by which the bar is retained within a recess of the clasp.

Preferably, the shell has a cut out portion towards the open side in a region of its wall that is located approximately in the same plane as the point where it is engaged by the clasp, for example approximately opposite to the midpoint of the bar. This can be of particular advantage when the bar can rotate relative to the clasp because it can reduce interference of the manipulator with rotation of the component and therefore allow rotation of the component through a larger angle.

Preferably, the manipulator includes a tube portion and a shaft which can rotate within the tube portion, the clasp being fastened to the shaft so that the component can be rotated relative to the tube portion. The assembly can include a handle which is provided by or fastened to the tube portion. The shaft can be driven by a rotary drive unit, especially when the component is a cutting tool.

Embodiments of the present invention will now be described by way of example with reference to the accompanying drawings in which:

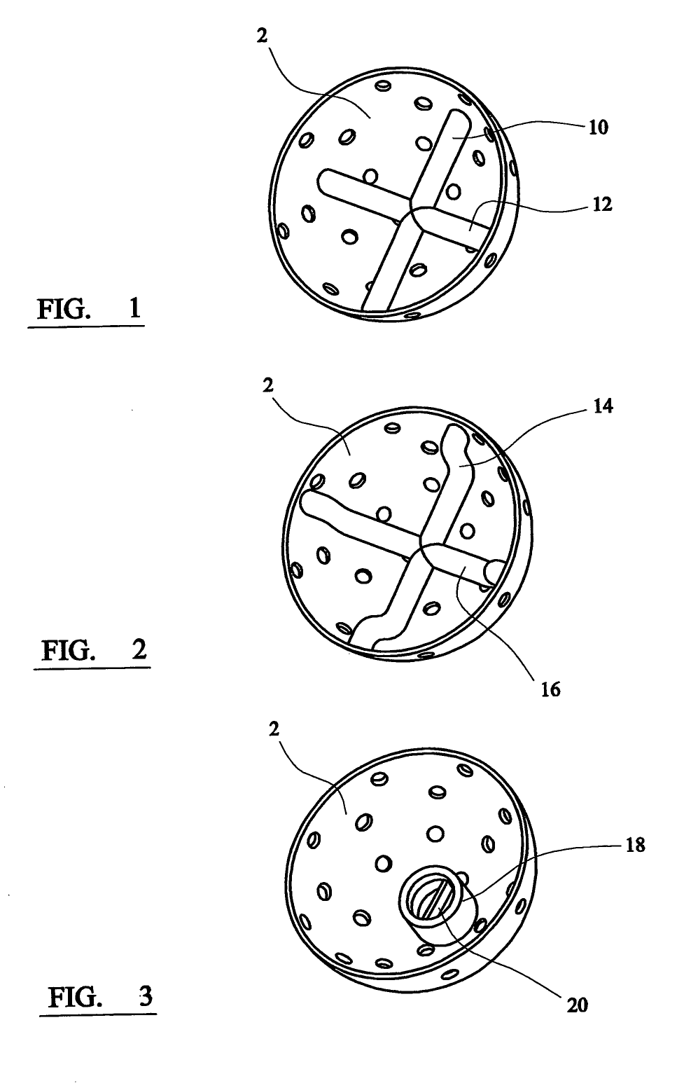

FIGS. 1 to 3 are isometric views of shell components of an assembly according to the invention.

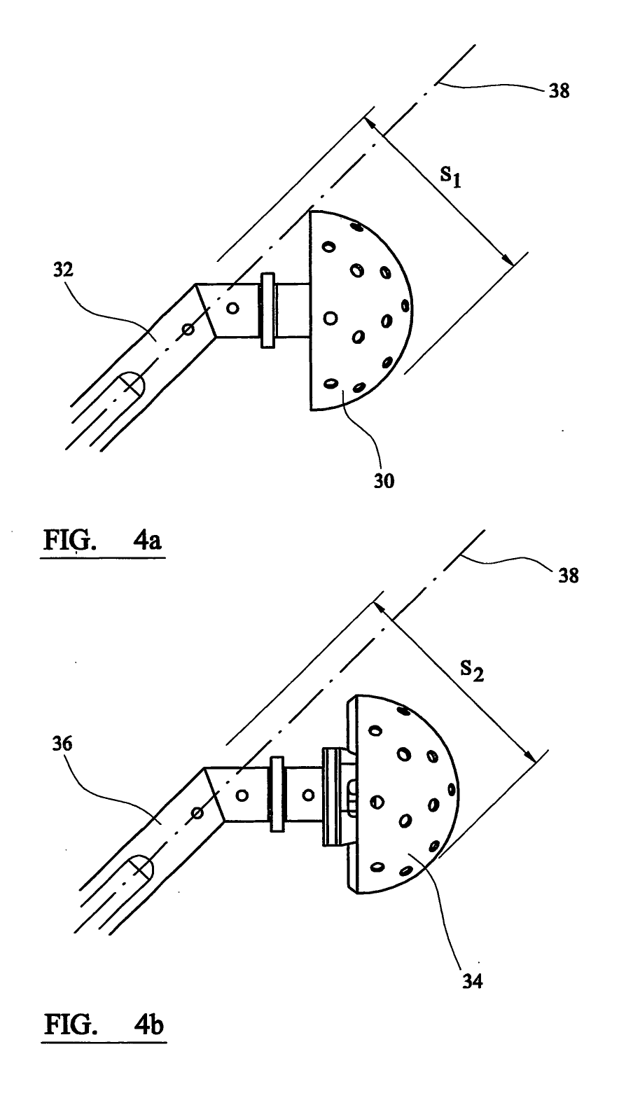

FIGS. 4a and 4b are side views of assemblies according to the present invention and as previously known.

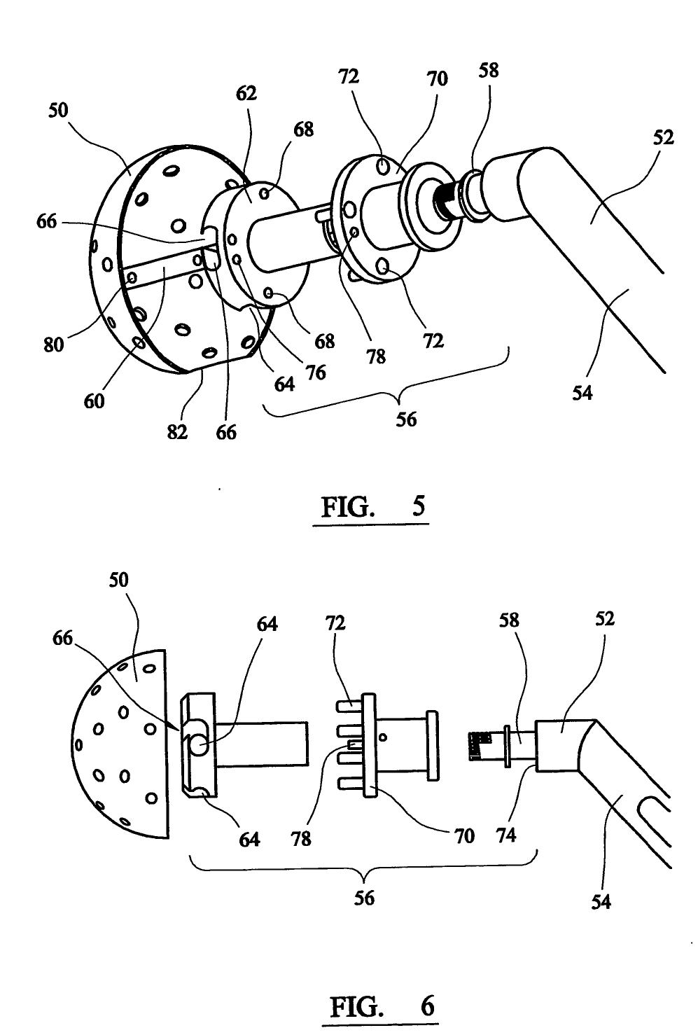

FIG. 5 is an exploded isometric view of an assembly according to the invention.

FIG. 6 is an exploded side view of the assembly shown in FIG. 5.

Referring to the drawings, FIGS. 1 to 3 show reamer components of an instrument set which can be used in the implantation of the acetabular cup component of a hip joint prosthesis. Each of the components comprises a hollow shell 2 formed from a suitable metallic material (for example a stainless steel) which has raised cutting teeth 4 arranged on its surface. The external surface of the shell defines a part of a sphere and the open side of the shell is at or slightly above about the equatorial plane of that sphere, so that the shell is symmetrical about the polar axis of the shell. Each of the cutting teeth is directed so as to cut the surface of a bone when rotated about the axis of symmetry. The shell is provided by a thin sheet of the metallic material, and can be manufactured by forming a sheet, or by other techniques such as casting and appropriate subsequent finishing.

Each of the reamer components shown in FIGS. 1 to 3 has at least one bar within it. The component shown in FIG. 1 has two bars 10, 12 which are fastened together at their centres on the axis of the shell. Each of the bars is fastened at its ends to the internal surface of the shell, for example by welding. Each of the bars is straight, when viewed along the axis of the sphere, and also when viewed from one side.

The component shown in FIG. 2 has two bars 14, 16 which, as with the component shown in FIG. 1, are fastened together at their centres on the axis of the shell. Each of the bars is cranked so that, when viewed from one side, the ends extend towards the open side of the shell, where they are connected to the shell by welding.

The component shown in FIG. 3 has a tube fixture 18 which is fastened to the internal surface of the shell at about the pole of the shell. The tube fixture has a bar 20 extends across the tube fixture, and which can be engaged by a clasp on a manipulator. Alternatively, the tube fixture can have other formations for engaging a clasp, such as bayonet formations or a thread.

In each of the embodiments shown in FIGS. 1 to 3, it is intended that the component be connected to a manipulator including a clasp which engages the fastening formation (which can be a bar, or another formation in the case of the component shown in FIG. 3) at about the point at which the fastening formation intersects the axis of the sphere. In each case, the part of the formation which is engaged by the clasp is located closer to the pole of the sphere than to the open side so that the clasp is located at least partially within the shell.

FIG. 4a shows an assembly according to the invention which includes a reamer component 30, which can be one of the reamers shown in FIGS. 1 to 3. The assembly includes a manipulator 32 which includes a clasp for engaging the bar within the reamer component shell.

FIG. 4b shown an assembly which also includes a reamer component 34 and a manipulator 36. The reamer heads and the manipulators shown in FIGS. 4a and 4b are identical apart from the fact that the bar in the reamer head shown in FIG. 4a is located within the shell, and the bar in the reamer head shown in FIG. 4b is located on the open side.

When the assemblies shown in FIGS. 4a and 4b are being used, they have to be inserted into a body cavity through an incision. The size of the assembly determines the size of the incision, measured perpendicular to the axis 38 of the manipulator. It can be seen that the size of the assembly shown in FIG. 4a (distance S₁) is smaller than the size of the assembly shown in FIG. 4b (distance S₂).

FIGS. 5 and 6 show an assembly according to the invention which comprises a reamer head 50 and a manipulator 52. The manipulator comprises a proximal portion 54 with which the assembly can be held. and a distal portion 56. The angle between the proximal and distal portions is about 135°. Each of the proximal and distal portions is hollow and a drive shaft 58 extends through them, via a universal joint at the point where the portions are joined.

The reamer head has a single bar 60 within it, extending between the opposite internal surfaces of the head (generally as one of the bars 10, 12 in the head shown in FIG. 1).

The manipulator includes a clasp head 62 which has four recesses 64 in it. Each of the recesses has an opening 66 on the face of the clasp head which is directed into the shell of the reamer head. The clasp head has a hole 68 extending through it associated with each of the recesses 64. A collar 70 which can slide relative to the clasp head along the distal portion of the manipulator has four closure pins 72 on it, directed along the axis of the distal portion, so that they can slide into and out of the holes 68 in the clasp head. The collar is resiliently biassed towards the clasp head by means of a spring acting against a biassing face 74.

One of the recesses 64 in the clasp head also has a lock pin hole 76 associated with it, extending through the clasp head into the opening. The collar 70 has a lock pin 78 on it, again directed along the axis of the distal portion, so that it can slide into and out of the lock pin hole in the clasp head. The lock pin 78 is shorter than the closure pins 72.

The bar 60 in the reamer head has a lock hole 80 within it towards one end thereof. The shell has a cut out portion 82 in its outer wall.

The manipulator shown in FIGS. 5 and 6 can be used with reamer heads which have two bars within them intersecting on the axis of the head as shown in FIGS. 1 and 2. This is possible by virtue of the arrangement of the four openings 66 and recesses 64 in the clasp head. However, when the reamer head only has one bar (as shown in FIG. 5), just two of the four openings 66 will be used.

In order to fasten the reamer head to the clasp head, the collar 70 is retracted along the distal portion 56 to withdraw the closure pins 72 and the lock pin 78 from their respective holes 68, 76 in the clasp head. The bar 60 in the reamer head is then inserted into the openings 66 in the clasp head which communicate with the recesses 64, and twisted relative to the clasp head so that the bar is received firmly within the recesses. Once the bar has been twisted clear of the openings 66, the collar 70 can be released so that it moves outwardly along the distal portion of the retractor, so that the closure pins 72 extend from their respective holes 68 in the clasp head, preventing the bar from inadvertently twisting out of the recesses.

When the lock pin hole 76 is aligned with the axis of the distal portion 56 of the manipulator, the collar can slide fully towards the reamer head so that the lock pin 78 extends through the lock pin hole 76 in the clasp head and into the lock hole 80 in the bar 60. This prevents rotation of the bar (and the reamer head) relative to the clasp and the manipulator. When the lock pin hole 76 is not aligned with the axis of the distal portion of the manipulator, the lock pin 78 is not able to extend from the lock pin hole in the clasp head, and this prevents movement of the collar fully towards the reamer head. However, because the lock pin 78 is shorter than the closure pins 72, the closure pins still serve to prevent the bar 60 from being moved out of the recesses in the clasp head.

The cut out portion 82 in the other wall of the reamer head allows the head to be rotated so that the plane containing the open side of the head is substantially parallel to the axis of the distal portion of the manipulator.

The collar can have more than one lock pin. For example, it can have two lock pins associated with opposite ones of the recesses. The or each lock pin need not be associated with a recess. For example, the lock pin can be located centrally on the collar.

The assembly of the present invention can be used to manipulate other components. For example, it can be used to manipulate instruments other than reamers. It can be used to manipulate implant components, for example the acetabular cup component of a hip joint prosthesis. Generally, the prosthesis will require a suitable mounting for the fastening formation.

1. An assembly for use in orthopaedic surgery, which comprises a component which is to be positioned within a body cavity to engage a bone, the component comprising a hollow shell which is open on one side to allow access to its interior and which has a fastening formation within it, and a manipulator having a clasp for engaging the fastening formation so as to fasten the component to the manipulator, in which the portion of the formation which is engaged by the clasp is located within the shell so that, when the component is fastened to the manipulator, the clasp is located at least partially within the shell.

2. An assembly as claimed in claim 1, in which the external surface of shell is symmetrical about an axis of rotation.

3. An assembly as claimed in claim 1, in which the external surface of the shell defines a part of a sphere.

4. An assembly as claimed in claim 2, in which the fastening formation intersects the axis of rotational symmetry at the point where it is engaged by the clasp.

5. An assembly as claimed in claim 1, in which the ratio of the depth of the fastening formation within the shell measured from the open side of the shell to the point where it is engaged by the clasp, to the length of the axis measured from the open side of the shell to the external surface of the shell opposite the open side, is at least about 0.2.

6. An assembly as claimed in claim 1, in which the fastening formation comprises a bar which extends across the shell.

7. An assembly as claimed in claim 6, in which the clamp defines at least one recess which can present a transverse opening which allows the bar to be positioned in the recess by sliding it transversely into the recess.

8. An assembly as claimed in claim 7, in which the clamp includes a locking part which can be moved between two positions in which (a) the bar is prevented from moving out of the recess, and (b) the bar can be moved out of the recess, respectively.

9. An assembly as claimed in claim 6, in which the bar is straight.

10. An assembly as claimed in claim 6, in which the bar is cranked.

11. An assembly as claimed in claim 1, in which the clasp allows rotation of the fastening formation.

12. An assembly as claimed in claim 11, in which the clasp has a lock which can engage the fastening formation to restrict rotation of the fastening formation relative to the clasp.

13. An assembly as claimed in claim 11, in which the shell has a cut out portion towards the open side in a region of its wall that is located approximately in the same plane as the point where it is engaged by the clasp.

14. An assembly as claimed in claim 1, in which the manipulator includes a tube portion and a shaft which can rotate within the tube portion, the clasp being fastened to the shaft so that the component can be rotated relative to the tube portion.

15. An assembly as claimed in claim 1, in which the component is a cutting tool, in which the external surface of the shell has cutting teeth.

16. An assembly as claimed in claim 1, in which the component is a component of an orthopaedic joint prosthesis.
